(19) United States
(12) Patent Application Publication
(10) Pub. No.: US 2024/0060102 A1
(43) Pub. Date: Feb. 22, 2024

(54) MICROORGANISM OIL COMPOSITION ENRICHED WITH DHA OR EPA DIGLYCERIDES

(71) Applicant: POLARIS, Quimper (FR)

(72) Inventors: Gildas BRETON, Quimper (FR); Lionel LARVOL, Quimper (FR)

(73) Assignee: POLARIS, Quimper (FR)

(21) Appl. No.: 18/271,705

(22) PCT Filed: Jan. 18, 2022
(86) PCT No.: PCT/EP2022/051011

§ 371 Date: Jul. 11, 2023

(30) Foreign Application Priority Data
Jan. 19, 2021 (FR) FR2100486

(51) Int. Cl. C12P 7/6472; C11C 1/04; C11C 1/10; C11C 3/06; C12N 9/20

(52) U.S. Cl. CPC C12P 7/6472; C11C 1/045; C11C 1/10; C11C 3/06; C12N 9/20; C12R 2001/72

(57) ABSTRACT

A microorganism oil composition enriched with eicosapentaenoic acid or with docosahexaenoic acid mainly in the form of diglycerides, and method for obtaining same. A microorganism oil composition includes an eicosapentaenoic acid and/or docosahexaenoic acid content greater than or equal to 500 mg/g of composition, and, with respect to the total quantity of glycerides, a quantity of diglycerides greater than 45% and a quantity of monoglycerides and diglycerides greater than 60%.

The present invention relates to a microorganism oil composition enriched with eicosapentaenoic acid or with docosahexaenoic acid mainly in the form of diglycerides, and method for obtaining same.

For several years now, one has been recommended to enrich one's diet with polyunsaturated fatty acids because of their demonstrated beneficial role in very many physiological reactions and pharmaceutical functions.

Among the polyunsaturated fatty acids of interest, there are those belonging to the omega-3 family such as eicosapentaenoic acid (EPA) and docosahexaenoic acid (DHA) and those belonging to the omega-6 family such as arachidonic acid (ARA).

DHA and EPA have been the subject of numerous physiological studies and their essential role in babies, children and adults is now known. DHA is thus known for its essential role in the development of the brain and retina and in the preservation of cognitive functions. As for EPA, this contributes to good cardiac functioning and has anti-inflammatory properties.

One way of increasing the intake of eicosapentaenoic acid (EPA) and of docosahexaenoic acid (DHA) in one's diet is to consume oil-based concentrates, in particular fish or microalga oils. The microalgae belonging to the Thraustochytrium or Schizochytrium genus are for example sources rich in omega-3 long-chain polyunsaturated fatty acids. The Schizochytrium sp microalga can produce between 20% and 35% docosahexaenoic acid, by total fatty acid weight (Hadley et al., 2017) (Hammond et al., 2001). In concentrates, the proportions of DHA and EPA have been increased compared with the proportions of natural oils.

The methods for concentrating, or enriching, oils with DHA or EPA firstly involve separating the fatty acids from the glycerol by ethanolic transesterification using a chemical catalyst or by enzymatic ethanolic transesterification. Secondly, enrichment with DHA or with EPA is implemented by molecular distillation, for separating the compounds according to their vapour tension, i.e. usually their molecular weight.

By way of illustration, the international patent application WO 2013/178936 will be cited, which describes a method for enriching with DHA in the form of ethyl esters a microalga oil belonging to the Thraustochytriales sp family.

In these concentrated oils of the prior art, the polyunsaturated fatty acids are in the form of triglycerides or in the form of ethyl esters.

Another important qualitative element of the oil, apart from the proportion of polyunsaturated fatty acids able to be obtained, is its bioavailability.

Thus several studies describe the interest of fatty acids in the form of diglycerides compared with the triglyceride form. Diglycerides consist of a glycerol skeleton on which two fatty acids are esterified. They may be in the form of three stereoisomers sn1,2, sn-1,3 or sn-2,3.

It has been demonstrated that adding fat in the form of diglycerides proved to be beneficial in dealing with illnesses such as obesity, cardiovascular illnesses and diabetes (Yee-Ying Lee et al.; Maki et al.; Saito et al.; Flickinger and Masuto; Prabhavathi Devi et al.) and more generally on nutritional prevention of metabolic syndrome.

Specifically, diglycerides provide:
Reduction in the blood cholesterol level,
Stimulation of beta oxidation,
Reduction in the accumulation of fats and reduction in body mass,
Modulation of glucose metabolism,
Improvement in bone health by increasing bone mineral density and improving the bone microstructure.

It is accepted that these effects are due to the very structure of the diglycerides. By comparison, numerous clinical studies, such as Tomonori Nagao et al., demonstrate that triglycerides, with a different structure, do not make it possible to obtain the effects mentioned above. This is because, owing to their structure, diglycerides use a digestion and absorption metabolic route different from that of other fats and in particular triglycerides.

For example, diglycerides ingested in sn-1,3 form are transformed into 1-(3) monoglycerides in the small intestine, which are very little re-esterified subsequently into triglycerides. By comparison, ingested triglycerides are transformed in to 2-monoglycerides, which are subsequently converted into triglycerides.

It is therefore clear that diglycerides prove to be interesting in the pharmaceutical, nutraceutical and food fields. In the field of food products, they could be used as a substitute for other less beneficial, or even harmful, fats, with regard to health.

It therefore seems necessary, and this is an aim of the invention, to be able to offer oils concentrated in DHA or EPA polyunsaturated fatty acids that are essentially in the form of diglycerides and have a high diglycerides content.

Moreover, the good bioavailability of long chain polyunsaturated fatty acids in the form of monoglycerides has also been verified (Valenzuleaa et al.). This study shows that supplementing with DHA monoglycerides makes it possible to have a DHA content in the plasma and red corpuscles higher than what is found when DHA is provided in the form of ethyl esters or in the form of triglycerides (Effect of Supplementation with Docosahexaenoic Acid Ethyl Ester and sn-2 Docosahexaenyl Monoacylglyceride on Plasma and Erythrocyte Fatty Acids in Rats, Ann Nutr Metab, 2005; 49: 49-53).

It therefore also seems advantageous to be able to offer oils concentrated in DHA or EPA polyunsaturated fatty acids that also have a high monoglyceride content.

The present invention thus relates to a microorganism oil composition enriched with polyunsaturated fatty acids, which is characterised in that it comprises:
an eicosapentaenoic acid and/or docosahexaenoic acid content greater than or equal to 500 mg/g of composition, and, with respect to the total quantity of glycerides:
a quantity of diglycerides greater than 45%,
a quantity of monoglycerides and diglycerides greater than 60%.

The composition according to the invention therefore has an excellent fatty acid profile with a high content of DHA or EPA polyunsaturated fatty acids in the form of diglycerides.

Within the meaning of the invention, “enriched with polyunsaturated fatty acids” means a composition comprising, after implementation of adapted concentration methods, a higher quantity, for example increased by a factor of 1.5 or 2, of polyunsaturated fatty acids than that which the composition had initially.

Equally, within the meaning of the invention, “total quantity of glycerides” means the sum of the quantities of monoglycerides, diglycerides, triglycerides and fatty acid ethyl esters.

The oil composition can be obtained from microalgae of the Thraustochytrium, Schizochytrium, Nannochloropsis, Isochrysis, Phaeodoctylum, Nitzchia, Staurosira, Crypthecodinium or Ulkenia genus.

The microalgae of the Thraustochytrium and Schizochytrium genera will preferentially make it possible to obtain an oil composition enriched with docosahexaenoic acid (DHA).

The microalgae of the Nannochloropsis, Isochrysis, Phaeodoctylum or Nitzchia genera will make it possible to obtain preferentially an oil composition enriched with eicosapentaenoic acid (EPA).

It should be noted that it is entirely possible to distinguish a microalga oil from an oil of another origin, in particular a fish oil, on the basis of its fatty acid profile. This is because microalga oils containing DHA are characterised by the presence of DPA n-6. This fatty acid is not present in fish oils for example, or very slightly in a quantity of less than 0.3% of the fatty acids. By way of example, a microalga oil with 400 mg of DHA/g of oil contains approximately 80 g of DPA n-6/g of oil. Microalga oils containing EPA are characterised by the absence of eicosenoic acid C20:1 (C20:1 W7 eicosenoic and C20:1 W9 gadoleic) and of docosenoic acid C22:1 (C22:1 W9 erucic and C22:1 W11 cetoleic). C20:1 et C22:1 are more specific to zooplankton and copepods in particular. Consequently, via the food chain, they are found in fish oils.

Advantageously, the composition comprises, with respect to the total quantity of glycerides:
a quantity of triglycerides of less than 30%,
a quantity of diglycerides of between 45 and 75%,
a quantity of monoglycerides of between 10 and 30%, Preferentially, the composition comprises, with respect to the total quantity of glycerides:
a quantity of triglycerides of between 10 and 30%,
a quantity of diglycerides of between 50 and 70%,
a quantity of monoglycerides of between 15 and 25%.

According to a first embodiment, the composition comprises a DHA content greater than or equal to 600 mg/g of composition, more preferentially greater than 700 mg/g of composition.

According to a second embodiment, the composition comprises an EPA content greater than or equal to 600 mg/g of composition, more preferentially greater than 700 mg/g of composition.

Another object of the invention is to propose a method for preparing a microorganism oil composition having the features described previously.

The invention therefore also relates to a method for preparing an oil composition enriched with omega-3 polyunsaturated fatty acids, characterised in that it comprises:
(a) a step of obtaining a microorganism oil comprising docosahexaenoic acid or eicosapentaenoic acid in the form of ethyl esters in a quantity greater than or equal to 500 mg/g of composition,
(c) a step of structuring the docosahexaenoic acid or the eicosapentaenoic acid mainly in the form of diglycerides by reaction between the oil and one or more enzymes in the presence of glycerol,
(d) a step of eliminating the glycerol,
(e) a step of short-path molecular distillation of the oil under vacuum.

Step a:

A microorganism oil comprising docosahexaenoic acid or eicosapentaenoic acid in the form of ethyl esters in a quantity greater than or equal to 500 mg/g of composition can for example be obtained commercially. For example, the oil sold under the name Omegavie Algae oils, by the company Polaris, France, will be cited.

Alternatively, an oil enriched with docosahexaenoic acid in the form of ethyl esters can be obtained by performing the following steps:
(i) a step of reaction between a microorganism oil comprising docosahexaenoic acid in the form of triglycerides and an alcohol in the presence of a chemical or enzymatic catalyst,
(ii) a step of increasing the concentration of docosahexaenoic acid by molecular distillation under high vacuum.

Step (i) is a transesterification step that will make it possible to obtain fatty acids in the form of ethyl esters (EE), which are then concentrated at the step (ii).

The starting oil at the step (i) may for example be a microalga oil of the genera Thraustochytrium or Schizochytrium.

An oil enriched with eicosapentaenoic acid in the form of ethyl esters can, for its part, be obtained by performing the following steps:
(i) a step of reaction between a microorganism oil comprising eicosapentaenoic acid and an alcohol in the presence of a chemical catalyst,
(ii) a step of concentrating and purifying the fatty acid ethyl esters including eicosapentaenoic acid ethyl esters by short-path molecular distillation under high vacuum.
(iii) a step of increasing the concentration of eicosapentaenoic acid by molecular distillation under high vacuum.

There again, step (i) is a transesterification step that will make it possible to obtain fatty acids in the form of ethyl esters (EE)).

The starting oil at the step (i) may for example be a microalga oil of the genera Nannochloropsis, Isochrysis, Phaeodoctylum or Nitzchia.

Alternatively again, it is possible, by implementing one and the same method, to obtain firstly an oil enriched with docosahexaenoic acid and secondly an oil enriched with eicosapentaenoic acid in the form of ethyl esters, by performing the following steps:
(i) a step of reaction between a microorganism oil comprising both docosahexaenoic acid and eicosapentaenoic acid, in the form of triglycerides, and an alcohol in the presence of a chemical or enzymatic catalyst,
(ii) a first step of molecular distillation under high vacuum of the oil resulting from the step (i), in a wiped-film evaporator coupled to a rectification column comprising at least seven theoretical plates, and recovery of a first residue and of a first distillate,
(iii) a second step of molecular distillation under high vacuum of the residue recovered at the step (ii), in said wiped-film evaporator coupled to the rectification column comprising at least seven theoretical plates, and recovery of a second residue and of a second distillate,

This is because some varieties of microalga, such as those of the Thraustochytrium and Schizochytrium genus, although containing mainly docosahexaenoic acid, may also contain not insignificant quantities of eicosapentaenoic acid. The above method allows the separation and concentration of eicosapentaenoic acid and of docosahexaenoic acid. The second distillate obtained contains EPA and the second residue contains DHA.

Step c:

The step (c) is implemented using oils with a concentration of DHA or EPA obtained at the step (a).

Preferentially, the structuring step (c) is performed at a molar ratio between docosahexaenoic acid or eicosapentaenoic acid and glycerol of 1.

Preferentially again, in the method according to the invention, the step (c) comprises:
the mixing, in a reactor under vacuum, under stirring and at a temperature of 35 to 40° C., of docosahexaenoic acid or eicosapentaenoic acid and of the enzyme or enzymes, and
additions of glycerol in several stages by portions, the additions being spaced apart by a period of between 1 hr and 3 hr.

The stirring parameter can be between 300 and 400 rev/min and the vacuum parameter can be below 15 mbar.

Very good results in terms of diglycerides content are obtained when the glycerol is added gradually.

The total quantity of glycerol will therefore be divided into three or four portions of identical quantity The first portion will be added when the fatty acids and the enzyme or enzymes are mixed, and then the other portions will be added one after the other by successive additions spaced apart by 1 to 3 hr, preferentially 2 hr.

The reaction can thus last for a total of between 4 and 10 hr.

According to a first embodiment, the enzyme used in the step (c) is a lipase B produced by Candida antartica. This enzyme is, for example, available commercially under the name Lipozyme 435®.

According to a second embodiment, the step (c) is performed by means of a mixture of two enzymes, the mixture being composed to the extent of 70-80% of a lipase B produced by Candida antartica and to the extent of 30-20% of a lipase produced by Thermomyces lanuginosus.

Very good results are in particular obtained with a mixture composed to the extent of 75% of lipase B produced by Candida antartica and to the extent of 25% of lipase produced by Thermomyces lanuginosus.

A lipase produced by Thermomyces lanuginosus is, for example, available commercially under the name Lipozyme TL IM®.

This enzyme, which is regioselective, is advantageous since it makes it possible to increase the level of diglycerides and, in particular, sn1-3 diglycerides. This is because, if EPA or DHA, molecules that take up a great deal of space, is fixed in position Sn-2 of the glycerol during a reaction initiated by the enzyme Candida antartica, for example in the form of Sn-2 monoglycerides, grafting a second fatty acid becomes more difficult, all the more so for forms with a high concentration of EPA or DHA. Adding the enzyme Thermomyces lanuginosus Sn-1, which is n-3 specific, in addition to the lipase B of Candida antartica, facilitates grafting of a second fatty acid on the glycerol.

The total quantity of enzymes used during the step (c) is of the order of 6 to 8%, preferentially 7.5%, with respect to the quantity of ethyl esters of the oil.

Step d:

The step (d) aims to eliminate the residual glycerol. It can be performed by settling.

Step e:

The step (e) is a step of short-path molecular distillation of the oil under vacuum that separates the molecules according to their molecular weight and thus concentrates the EPA or DHA mainly in the form of diglycerides.

Preferably, this step takes place in a short-path still at a wall temperature above 150° C., for example between 160 and 220° C., and a vacuum below 0.02 mbar.

Step b:

According to a particular embodiment, the method comprises, between the step (a) and the step (c):
a step (b) of structuring the docosahexaenoic acid or the eicosapentaenoic acid in the form of free fatty acids.

This structuring step is a saponification reaction that results in the formation of free fatty acids that are then used in the subsequent step (c).

This embodiment advantageously makes it possible to obtain, at the end of the method according to the invention, higher diglyceride and monoglyceride levels than the embodiments in which the step (b) is not performed.

Advantageously, according to this particular embodiment, the method comprises, following the step (e) of short-path molecular distillation:
a step (f) of recovery of a fraction comprising docosahexaenoic acid or eicosapentaenoic acid in free form and in the form of monoglycerides, and of reintroduction of this fraction at the step (c).

The invention also relates to an oil composition, characterised in that it comprises a mixture of an oil composition as defined previously, or obtained by a method as described previously, and at least one other oil.

An oil composition according to the invention can in fact be mixed with other oils such as vegetable oils or microorganisms, having other qualitative characteristics. For example, it will be possible to mix an oil enriched with EPA essentially in the form of glycerides and obtained according to the invention with an oil enriched with DHA in the form mainly of triglycerides or ethyl esters. Other mixtures are naturally possible.

The invention also relates to a food supplement, characterised in that it comprises an oil as defined previously or obtained by a method as described previously.

A food supplement may be in any form, such as a capsule, tablet, a liquid or a gel.

The invention also relates to a food product, characterised in that it comprises an oil composition as defined previously or obtained by a method as described previously.

A food product may be any product intended as food, in any form. This may in particular be a nutritional formulation such as a baby or dietary preparation.

The invention also relates to a pharmaceutical or nutraceutical composition, characterised in that it comprises an oil composition as defined previously or obtained by a method as described previously.

Finally, the invention relates to an oil composition as defined previously or obtained by a method as defined previously, for use thereof in preventing and/or treating obesity, overweight or bone illnesses, or in reducing cholesterol levels and modulating glucose metabolism.

The invention will be best understood from the reading of the example embodiments that follow.

### EXAMPLE 1: OBTAINING MICROALGA OILS ENRICHED WITH DHA OR EPA MAINLY IN THE FORM OF DIGLYCERIDES

Step (a) Obtaining Microalga Oils Comprising DHA or EPA Mainly in the Form of Ethyl Esters

1) Obtaining Microalga Oil Comprising DHA: H1(DHA) Oil

The starting oil is a raw oil produced by the Schizochytrium sp T18 microalga strain sold by Mara Renewables Corporation. It initially contains 329 mg/g of DHA.

Step (i): Transesterification

A transesterification reaction is performed on a biomass of 1800 kg of microalga oil using 450 kg of ethanol and 21.6 kg of sodium ethylate, in a suitable reactor.

The reaction temperature is 50° C. and the reaction time is 1 hr. At the end of the reaction, the excess ethanol is evaporated under vacuum, and then the mixture is cooled to a temperature of approximately 30° C. and then subjected to settling for 1 hr. The light phase is recovered and then the glycerol is drained off. A second settling is performed for 30 sec. The glycerol and the residual monoglycerides are drained off.

A washing with acidic water is next performed by adding 17% demineralised water containing 2.5% phosphoric acid (75%) under stirring for 20 sec. The mixture is settled for 20 sec and the aqueous phase is drained off. There follows drying under vacuum (pressure <90 mbar) at 60° C. for a time greater than 2 hr.

At the end of this step, the oil contains 329 m/g of DHA in the form of ethyl esters.

Step (ii): Concentration of DHA by Molecular Distillation

The oil is next conducted into a degasser and then passes through a wiped-film evaporator.

The vapours are next distilled through a rectification column that is coupled to the evaporator supplied by the company UIC GmbH. A reflux of the distillate, i.e. a reintroduction of the distillate into the column, can increase the separative efficacy of the latter. The column used contains approximately seven theoretical plates. The distillation residue is recovered and represents the fraction enriched with DHA.

The operational conditions are as follows: T° of the evaporator: 225° C.; vacuum of the rectification column: 0.1 mbar; reflux ratio 75%, T° (column top): 195° C.

The quantity of DHA in the form of ethyl esters is 758 mg/g.

2) Obtaining Microalga Oil Comprising EPA: H2(EPA) Oil

The starting oil is a raw oil produced by the Nannochloropsis sp strain obtained after culture of the biomass for about a week under photoautotrophic conditions. The fat is extracted from the biomass by ethanolic maceration. After separation of the remaining solid phase, the ethanol is evaporated under vacuum.

The EPA produced by photoautotrophy is incorporated in the membrane lipids, either in the form of glycolipids, or in the form of phospholipids, or in the form of free fatty acids. The triglyceride form is little represented.

Step (i): Transesterification

A transesterification reaction is performed on a biomass of 100 kg of microalga oil using approximately 200 kg of ethanol and 10 kg of sulphuric acid, in a suitable reactor (reflux 60° C. >10 hr). The transesterification is performed acidically, with sulphuric acid.

Step (ii): Concentration of EPA by Short-Path Molecular Distillation Under Vacuum

The conditions are a wall temperature of the evaporator of between 175 and 180° C., and a vacuum below 0.1 mbar.

Step (iii): Concentration of EPA by Molecular Distillation on Rectification Column.

The operational conditions are as follows: T of the evaporator: 230° C.; vacuum of the rectification column: 0.07 mbar; reflux ratio 65%, T° (column top): 107° C., T° (column bottom): 147° C.

The quantity of EPA in the form of ethyl esters is 735 mg/g.

3) Obtaining Microalga Oil Comprising DHA and Microalga Oil Comprising EPA Using One and the Some Method: H3(DHA) Oil and H4(EPA) Oil

The starting oil is a raw oil produced by the Schizochytrium sp microalga strain sold by the company DSM under the trade name Life's DHA 60. It initially contains 360 mg/g of DHA and 184 mg/g of EPA.

Step (i): Transesterification

A transesterification reaction is performed on a biomass of 19 kg of microalga oil using 4.75 kg of ethanol and 222 kg of sodium ethylate, in a suitable reactor.

The reaction temperature is 50° C. and the reaction time is 1 hr. At the end of the reaction, the excess ethanol is evaporated under vacuum, and then the mixture is cooled to a temperature of approximately 30° C. and then subjected to settling for 1 hr. The light phase is recovered and then the glycerol is drained off. A second settling is performed for 30 sec. The glycerol and the residual monoglycerides are drained off.

A washing with acidic water is next performed by adding 3.2% demineralised water containing 76.4% phosphoric acid (75%) under stirring for 20 sec. The mixture is settled for 20 sec and the aqueous phase is drained off. There follows drying under vacuum (pressure <90 mbar) at 60° C. for a time greater than 2 hr.

At the end of this step, the oil contains EPA and DHA in the form of ethyl esters.

Step (ii): First Molecular Distillation Step

The oil is next conducted into a degasser and then passes through a wiped-film evaporator. The vapours are next distilled through a rectification column that is coupled to the evaporator supplied by the company UIC GmbH. The aim is here to eliminate the lightest fatty acids while keeping the DHA and EPA. The column used contains seven theoretical plates. The distillation residue is recovered and represents the fraction enriched with EPA and DHA.

The operational conditions are as follows: T° of the evaporator: 225° C.; vacuum of the rectification column: less than 0.1 mbar; reflux ratio 70%, T° (column bottom): 190° C., T° (column top): 135° C.

At the end of this step, a residue fraction and a distillate fraction are obtained. The residue fraction contains EPA and DHA.

Step (iii): Second Molecular Distillation Step

The operation of the step (II) performed again on the residue. The latter is therefore conducted again into the degasser and then passes through the wiped-film evaporator. The vapours are next distilled through the rectification column coupled to the evaporator as in the case of the previous step (ii). The aim is here to separate the DHA and the EPA.

The operational conditions are as follows: T° of the evaporator: 235° C.; vacuum of the rectification column: less than 0.05 mbar; reflux ratio 60%, T° (column bottom): 202° C., T° (column top): 160° C.

At the end of this step, a distillate fraction containing 733 mg/g of EPA and a residue fraction containing 706 mg/g of DHA are obtained.

The H1(DHA), H2(EPA), H3(DHA) and H4(EPA) oils thus all contain an amount of EPA or DHA greater than 700 mg/g of oil and are ready to be used in the following steps.

Step (c): Structuring the Docosahexaenoic Acid or the Eicosapentaenoic Acid Mainly in the Form of Diglycerides by Reaction Between the Oil and One or More Enzymes in the Presence of Glycerol

Option 1: Reaction in the Presence of a Single Enzyme

Raw materials and enzyme:
180 g of an oil obtained at the step (a),
4 portions each of 12.5 g of glycerol (100%), i.e. 50 g of glycerol in total,
13.5 g of Candida antartica lipase B (Upozyme 435®),

Molar ratio between docosahexaenoic acid or eicosapentaenoic acid and glycerol of 1.

180 g of oil and 12.5 g of glycerol were mixed in a reactor, with the enzyme. The mixture was heated at 37° C., stirred at 330 rev/min and placed under vacuum. The rest of the glycerol was added in four stages, i.e. 12.5 g every two hours. The vacuum was moderate during the first eight hours of the reaction, i.e. approximately 15 mbar, then high, i.e. below 5 mbar.

Option 2: Reaction in the Presence of a Mixture of Enzymes

The step (c) is performed in the same way as described previously except for the fact that the following mixture of enzymes is used.

10.1 g of Candida antartica lipase B (Lipozyme 435®),

3.4 g of Thermomyces lanuginosus lipase (Lipozyme TL IM®).

Step (d) Elimination of the Glycerol

The glycerol is eliminated by settling.

Step (e) Short-Path Molecular Distillation of the Oil Under Vacuum

The conditions are a wall temperature of the evaporator of between 160 and 220° C., and a vacuum below 0.02 mbar.

Results

At the end of the method, oils are obtained having more than 700 mg/g of DHA or EPA according to the following glyceride profile, expressed as a percentage with respect to the quantity of total glycerides (analysis performed by gas chromatography) (Table 1):

TABLE 1

| | | | Step (c) with Candida | |
|---|---|---|---|---|
| | | | antartica lipase B and | |
| | Step (c) with Candida | | Thermomyces lanuginosus | |
| | antartica lipase B | | lipase | |
| | Profile of the glycerides | | Profile of the glycerides | |
| Starting oil at the step a) | at the end of the step (e) | | at the end of the step (e) | |
| Oil containing more than | 55% | DG | | |
| 700 mg/g of DHA in the | 15% | MG | | |
| form of ethyl esters | 30% | TG | | |
| H1(DHA) or H3(DHA) | | | | |
| Oil containing more than | 50% | DG | 52% | DG |
| 700 mg/g of EPA in the | 22% | MG | 22% | MG |
| form of ethyl esters | 25% | TG | 23% | TG |

H2(EPA) or H4(EPA)

As a Percentage of the Total Quantity of Monoglycerides, Diglycerides, Triglycerides and Fatty Acid Ethyl Esters; MG: Monoglycerides; DG: Diglycerides; TG: Triglycerides

In all cases, the EPA or DHA fatty acids are mainly in the form of diglycerides. It should be noted that the use of the combination of enzymes makes it possible to increase the diglyceride content if the results obtained are compared with the starting oils H2(EPA) and H4(EPA) using at the step c) one enzyme versus a mixture of two enzymes. Furthermore, the reaction was more rapid, which is advantageous.

The total diglyceride and monoglyceride contents are high, with, for each oil, a quantity greater than 70%.

### EXAMPLE 2: OTHER EXAMPLE OF OBTAINING A MICROALGA OIL ENRICHED WITH DHA MAINLY IN THE FORM OF DIGLYCERIDES

This example takes place in the same way as example 1, starting at the step a) with an H1(DHA) or H3(DHA) oil, except for the fact that the method performs, between the step (a) and the step (c), a step (b) and a step (f) after the step (e).

Step b) Structuring the Docosahexaenoic Acid or the Eicosapentaenoic Acid in the Form of Free Fatty Acids

For each oil resulting from the step a), a saponification reaction intended to obtain the fatty acids in free form is implemented

Step c): takes place in an identical manner to what was described in example 1.

At the end of the step c) the oil contains 57% diglycerides, 24% monoglycerides, 8% triglycerides and 11% free fatty acids.

Step d): takes place in an identical manner to what was described in example 1.

Step e): takes place in an identical manner to what was described in example 1.

Step f): performance of the step e) enables a fraction containing DHA to be recovered in the form of monoglycerides and in free form, which is re-conveyed and reintroduced at the step c).

Results

At the end of the method, oils are obtained having more than 700 mg/g of DHA or EPA according to the following glyceride profile, expressed as a percentage with respect to the quantity of total glycerides (analysis performed by gas chromatography) (Table 2):

TABLE 2

| | Step (c) with Candida antartica | |
|---|---|---|
| | lipase B | |
| | Profile of the glycerides at the | |
| Starting oil at the step a) | end of the step (e) | |
| Oil containing more than 700 mg/g of | 68% | DG |
| DHA in the form of ethyl esters | 22% | MG |
| H1(DHA) or H3(DHA) | 10% | TG |

As a Percentage of the Total Quantity of Monoglycerides, Diglycerides, Triglycerides and Fatty Acid Ethyl Esters; MG: Monoglycerides; DG: Diglycerides; TG: Triglycerides

The DHA fatty acids are mainly in the form of diglycerides.

The total diglyceride and monoglyceride contents are high, with, for each oil, a quantity of 90%.

1. A microorganism oil composition enriched with polyunsaturated fatty acids, wherein the microorganism oil composition comprises:
an eicosapentaenoic acid and/or docosahexaenoic acid content greater than or equal to 500 mg/g of composition,
and, with respect to the total quantity of glycerides:
a quantity of diglycerides greater than 45%,
a quantity of monoglycerides and diglycerides greater than 60%.

2. The microorganism oil composition according to claim 1, wherein the microorganism oil composition comprises, with respect to the total quantity of glycerides:
a quantity of triglycerides of less than 30%,
a quantity of diglycerides of between 45 and 75%,
a quantity of monoglycerides of between 10 and 30%.

3. The the microorganism oil composition according to claim 1, wherein the microorganism oil composition comprises, with respect to the total quantity of glycerides:
a quantity of triglycerides of between 10 and 30%,
a quantity of diglycerides of between 50 and 70%,
a quantity of monoglycerides of between 15 and 25%.

4. The the microorganism oil composition according to claim 1, wherein the microorganism oil composition comprises a docosahexaenoic acid content greater than or equal to 600 mg/g of composition, more preferentially greater than 700 mg/g of composition.

5. The the microorganism oil composition according to claim 1, wherein the microorganism oil composition comprises an eicosapentaenoic acid content greater than or equal to 600 mg/g of composition.

6. A method for preparing an oil composition enriched with omega-3 polyunsaturated fatty acids, wherein that the method comprises:
(a) a step of obtaining a microorganism oil comprising docosahexaenoic acid or eicosapentaenoic acid in the form of ethyl esters in a quantity greater than or equal to 500 mg/g of composition,
(c) a step of structuring the docosahexaenoic acid or the eicosapentaenoic acid mainly in the form of diglycerides by reaction between the oil and one or more enzymes in the presence of glycerol,
(d) a step of eliminating the glycerol,
(e) a step of short-path molecular distillation of the oil under vacuum.

7. The method according to claim 6, wherein the step (c) is performed at a molar ratio between docosahexaenoic acid or eicosapentaenoic acid and glycerol of 1.

8. The method according to claim 6, wherein the step (c) comprises:
the mixing, in a reactor under vacuum, under stirring and at a temperature of 35 to 40° C., of docosahexaenoic acid or eicosapentaenoic acid and of an enzyme or enzymes, and
additions of glycerol in several stages by identical portions, the additions being spaced apart by a period of between 1 hr and 3 hr.

9. The method according to claim 6, wherein the step (c) is performed using a lipase B produced by Candida antartica.

10. The method according to claim 6, wherein the step (c) is performed by means of a mixture of two enzymes, the mixture being composed to the extent of 70 to 80% of a lipase B produced by Candida antartica and to the extent of 30-20% of a lipase produced by Thermomyces lanuginosus.

11. The method according to claim 6, wherein the method comprises, between the step (a) and the step (c):
(b) a step of structuring the docosahexaenoic acid or the eicosapentaenoic acid in the form of free fatty acids.

12. The method according to claim 11, wherein the method comprises, following the step (e):
(f) a step of recovery of a fraction comprising docosahexaenoic acid or eicosapentaenoic acid in free form and in the form of monoglycerides, and of reintroduction of this fraction at the step (c).

13. An oil composition that comprises a mixture of an oil composition as defined in claim 1.

14. A food supplement that comprises an oil composition as defined in;
a microorganism oil composition enriched with polyunsaturated fatty acids, wherein it comprises:
the eicosapentaenoic acid and/or docosahexaenoic acid content greater than or equal to 500 mg/g of composition,
and, with respect to the total quantity of glycerides:
the quantity of diglycerides greater than 45%,
the quantity of monoglycerides and diglycerides greater than 60%,
or obtained by the method according to claim 6.

15. A food product that comprises an oil composition as defined in;
a microorganism oil composition enriched with polyunsaturated fatty acids, wherein it comprises:
the eicosapentaenoic acid and/or docosahexaenoic acid content greater than or equal to 500 mg/g of composition,
and, with respect to the total quantity of glycerides:
the quantity of diglycerides greater than 45%,
the quantity of monoglycerides and diglycerides greater than 60%,
or obtained by a method according to claim 6.

16. A pharmaceutical or nutraceutical composition; that comprises an oil composition as defined in:
a microorganism oil composition enriched with polyunsaturated fatty acids, wherein it comprises:
the eicosapentaenoic acid and/or docosahexaenoic acid content greater than or equal to 500 mg/g of composition,
and, with respect to the total quantity of glycerides:
the quantity of diglycerides greater than 45%,
the quantity of monoglycerides and diglycerides greater than 60%,
or obtained by a method according to claim 6.

17. An oil composition as defined in a microorganism oil composition enriched with polyunsaturated fatty acids, wherein it comprises:
the eicosapentaenoic acid and/or docosahexaenoic acid content greater than or equal to 500 mg/g of composition,
and, with respect to the total quantity of glycerides:
the quantity of diglycerides greater than 45%,
the quantity of monoglycerides and diglycerides greater than 60%,
or obtained by a method according to claim 6, for use thereof in preventing and/or treating obesity, overweight or bone illnesses, or in reducing cholesterol levels and modulating glucose metabolism.

18. An oil composition that comprises a mixture of an oil composition obtained by a method according to claim 6, and of at least one other oil.
